(19) United States
(12) Patent Application Publication
(10) Pub. No.: US 2023/0218166 A1
(43) Pub. Date: Jul. 13, 2023

(54) OPHTHALMIC PORTABLE LASER SLIT LAMP AND METHOD FOR EYE INSPECTION

(71) Applicant: SZUSTER HOLDINGS LLC, Chicago, IL (US)

(72) Inventors: Nestor Mario SZUSTER, Capital Federal, Buenos Aires (AR); Silvio ROSINOV, Buenos Aires City (AR)

(73) Assignee: SZUSTER HOLDINGS LLC, Chicago, IL (US)

(21) Appl. No.: 17/996,279

(22) PCT Filed: Apr. 17, 2020
(86) PCT No.: PCT/US2020/028728

§ 371 Date: Oct. 14, 2022

(51) Int. Cl. A61B 3/135; A61B 3/14; A61B 3/00; A61B 3/107; A61B 3/10; A61B 3/117; H01S 5/024; H01S 5/042; H01S 5/0239; H01S 5/02253

(52) U.S. Cl. CPC A61B 3/135; A61B 3/14; A61B 3/0033; A61B 3/107; A61B 3/1005; A61B 3/1173; H01S 5/02469; H01S 5/042; H01S 5/0239; H01S 5/02253; A61B 2560/0431; A61B 2560/028; A61F 2009/00853

(57) ABSTRACT

An ophthalmic portable laser slit lamp for ophthalmic examination and a method of eye inspection. The device comprises a portable housing containing an electronic timer circuit, a rechargeable battery, a laser module containing a laser emitting diode, a fixed focusing lens that sets the appropriate focal distance for the examination method and a line generator lens acting as a slit aperture. The laser beam aimed to the eye of the patient illuminates the eye with a very thin straight laser line at a fixed focal distance. The device also comprises a safety timer circuit that protects the patients eye against irradiation overload. The method of the invention allows the surgeon to detect surgical eye disorders at the operating room and helps to carry out a correct diagnosis in a much more precise and effective way than any light or laser spot device.

### TECHNICAL FIELD OF THE INVENTION

The present invention relates to the technical field of medical equipment, in particular to an ophthalmic portable laser slit lamp and a method of eye inspection.

### BACKGROUND OF THE INVENTION

In ophthalmology, optically reflected corneal images are commonly used as a diagnostic tool to identify various eye diseases. The conventional LED and halogen slit lamp microscope is a conventional ophthalmic optical inspection tool. It enables the doctor to observe the superficial lesions of the human eye, but also can clearly show the lesions in the deep tissues of the eyes. With the aid of the slit image, structures and layers in the eye can be better recognized during diagnostic and surgical procedures. Surgical slit lamps are used for the most part in combination with a surgical microscope in intraocular and especially in retinal surgery. By means of the illuminated field on the retina, membranes that have become diseased, which are very thin but nevertheless greatly reduce the patient's sight, can be detected early and successfully operated on.

### PRIOR ART

Most existing slit lamps are relatively bulky, and are usually fixed indoors for eye examination, being inconvenient to carry, and having a limited application range. Surgical slit lamps of this kind are on the market, for example, under the name “Leica® Slit Illuminator,” and are described in Leica® web page www.Leica-microsystems.com/products/surgical-microscopes/p/leica-m822-f20/gallery/

Other existing surgical slit lamps such as (i) Ray Vision® portable slit lamp (www.lunar-health.com) or (ii) Digital Eye center® S150 Ultra Potable Slit Lamp® (https://www.digitaleyecenter.com/product/ultra-portable-slit-lamp-s150/) are portable but show the following disadvantages: a. they use conventional LEDs for light generation instead of laser diodes, b. they lack of a fixed focus, c. they comprise no internal safety timers and d. they lack of a fixed slit.

Document CN 208610812 discloses a pen type slit lamp with magnifying glass for the eye examination, and a casing upper end connected with a volume bracket that is used for holding the patient's forehead. The lighting system has a slit for emitting light to the patient's eye. The magnifying glass is used for observing the patient's eye on the casing. The pen type slit lamp magnifying glass has five kinds of light fence pieces available, for providing omni-directional inspection. However, the document fails to describe the main features of the present invention, namely, it uses a conventional LED for light generation instead of a laser diode. Also its focus is not fixed; it does not comprise internal safety timer and its slit is not fixed; all of these features being essential for assuring safety for the patient's eye.

Document U.S. Pat. No. 8,277,047 B2 describes an illumination unit for the generation of optical sectional images in transparent media, particularly in the eye. In the arrangement, the low-divergence beams emitted by a laser serving as illumination source are imaged on or in the eye under examination by a reflection element which is controllable in a defined manner and beam deflection elements present in the beam path. The optical sectional images resulting in and on the eye can be observed and/or recorded, further processed and evaluated with an image processing unit in a known manner. In the solution according to the document, a sectional image is generated by the deliberate periodic beam deflection of a particularly fine laser beam with high depth of focus, which sectional image remains sharp through the entire dimension of the object to be examined and makes possible an improved evaluation. The intensity of the laser beam bundle can be varied in such a way that it is sufficient for observation and documentation, but so that the diameter of the beam bundle is fine enough for a high detail resolution. However, the unit of the cited document is not portable. It is mainly designed for carrying out surgical incisions in a sterilized environment and it has to be used together with a surgical microscope that amplifies the image by means of its slit lamp binoculars. Also, due to the way it is used, the unit comprises no internal safety timer.

Document U.S. Pat. No. 7,724,429 B2 relates to a microscope (10) having an illumination apparatus (26) having a light source (1) and an optical system. The light source (1) is embodied to output a coherent light beam bundle along a defined illumination beam path (2a), and the optical system in the illumination beam path (2a) encompasses a spatial light modulator (3) for modifying the illuminated field (4). A surgical microscope (10) is preferably equipped with an illumination apparatus (26) of this kind that is arranged adjustably in two directions on the surgical microscope (10). However, the unit of the cited document is not portable and, unlike the present invention, it cannot be used with any other microscope. Also, due to the way it is used, the unit comprises no internal safety timer.

Document US describes a laser collimator that includes an optical lens having an optical axis; a sliding lens holder for housing the lens having a longitudinal opening parallel to the optical axis; a stationary lens holder ring for fitting to the front of the sliding lens holder and having a protruding key for receiving the longitudinal opening; and a focusing ring movable along the optical axis, wherein the protruding key placed within the longitudinal opening prevents the sliding lens holder from rotating. However, the document is aimed to another usage, namely for geometric alignment of telescope focusing and, since it is not to be aimed into an eye, it includes no safety timer.

Document US20150085254 describes methods and apparatuses for a micro-display based slit lamp illumination system. A first optical element is configured to generate a micro-display image including an illuminated area. A second optical element is configured to receive the micro-display image, and focus the micro-display image upon an eye to be examined, wherein light is reflected from the eye as a result of the illuminated area. However, the principles of the cited document are different from those of the present invention which does not generate an illuminated are but a fine light line instead. Also, the apparatus of the cited document is not portable and does not comprise an internal safety timer.

Therefore, since the known non-portable slit lamps use halogen lamps and the known portable slit lamps use LEDs, it is apparent that none of the prior art slit lamps (portable or desk-type) use laser diodes for generating the required light for a correct eye inspection. Through medical practice has proven that laser light has better illuminating power than halogen and conventional LEDs in this field and is much less damaging to the eye.

Also, none of the prior art documents mention the use of safety feature such as an internal safety timer that will set a time limit to laser exposure of the eye. Also, none of the prior art documents mentions or suggests that the focus is fixed and that the slit is fixed (not capable of being broadened) which guarantees a very detailed vision of the surgical defects.

### SUMMARY OF THE INVENTION

The technical problem mainly solved by the present invention is to provide a safe portable laser slit lamp that overcomes the above-mentioned drawbacks in the prior art and a method of eye inspection using the new laser slit lamp.

In order to solve the above technical problem of the state of the art, the technical solution offered by the present invention is to provide an ophthalmic portable laser slit lamp for ophthalmic examination and a method of eye inspection. The device comprises a portable housing containing an electronic timer circuit, a rechargeable battery, a laser module containing a laser emitting diode, a fixed focusing lens that sets the appropriate focal distance for the examination method and a line generator lens acting as a slit aperture. The laser beam aimed to the eye of the patient illuminates the eye with a very thin straight laser line at a fixed focal distance. The device also comprises a safety timer circuit that protects the patient's eye against irradiation overload. The method of the invention allows the surgeon to detect surgical eye disorders at the operating room and helps to carry out a correct diagnosis in a much more precise and effective way than any light or laser spot device.

### BRIEF DESCRIPTION OF THE DRAWINGS

FIGS. 1A-1B are schematic upper views showing the housing of the portable laser slit lamp of the present invention and FIG. 1C show a bottom view of the housing.

FIG. 2A is aside view and FIG. 2B is a front view of the housing.

FIG. 2C is an inner view of the portable laser slit lamp of the present invention;

FIG. 3A is an upper perspective schematic view of the circuital elements of the timer circuit of the present invention and FIG. 3B shows the way the timer circuit is mounted on the housing.

FIG. 4 is a schematic drawing showing the timer circuit of the present invention.

FIGS. 5A and 5B show a side view and a perspective front view of the laser diode module of the present invention.

FIG. 6 shows a block schematic of the laser diode module of the present invention.

FIG. 7 shows a top plan view of the placement of the circuit elements of the timer circuit of the present invention.

FIGS. 8A and 8B show photos of a donor scroll of a research cornea orientated in a correct position prepared for a DMEK surgery.

FIGS. 9A and 9B show photos of a donor scroll of a research cornea in an upside down position prepared for the DMEK surgery.

FIGS. 10A and 10B show the interoperative photos of a donor scroll in an upside down position inside a recipient eye.

FIGS. 11A-11B show photos of the scroll shown in FIGS. 10A and 10B after being turned over inside the eye in the same surgery.

FIGS. 12A-12B show photos of a scroll in another cornea with correct orientation in another surgery.

FIG. 13A shows a photo at the left of the gap between the donor and the recipient in a DSAEK surgery (Descemet Stripping Automated Endothelial Keratoplasty) and at the right a comparative image with an OCT (Optical Coherence Tomograph).

FIG. 13B shows a photo at the left of a perfect adherence of the donor graft after rebubbling and at the right a corresponding image of an OCT.

FIG. 14A shows a photo at the left of an OCT image of a chronic Descemet detachment, and at the right the same image using the laser lamp of the present invention.

FIG. 14B shows a photo at the right of the image formed by the laser lamp of the present invention after solving the eye's issue.

FIG. 15A shows a photo at the left of a stromal detachment after a complicated DALK (Deep Anterior Lamellar Keratoplasty) when using the laser lamp of the present invention in an interoperative view, and at the right an OCT image of the same detachment.

FIG. 15B shows a photo at the right of an interoperative image of a completely attached stroma using the laser lamp of the present invention, and at the left an image after solving the eye's issue.

FIG. 16A shows a photo of an image of an ICL (Implantable contact lens) implanted with an uneven vault using the laser lamp of the present invention.

FIG. 16B shows a photo of a digital image of the uneven gap of FIG. 16A.

FIG. 16C shows a photo of an ICL inside the eye; the right arrow pointing at the anterior surface of the ICL and the left arrow pointing at the posterior surface of the ICL.

### DETAILED DESCRIPTION OF THE INVENTION

The preferred embodiments of the present invention are described in detail below with reference to the enclosed drawings, so that the advantages and features of the present invention can be more easily understood by those skilled in the art.

The present invention provides a portable laser slit lamp, which is used for ophthalmic examination, having a small size, light weight, convenient simple operation, and low cost.

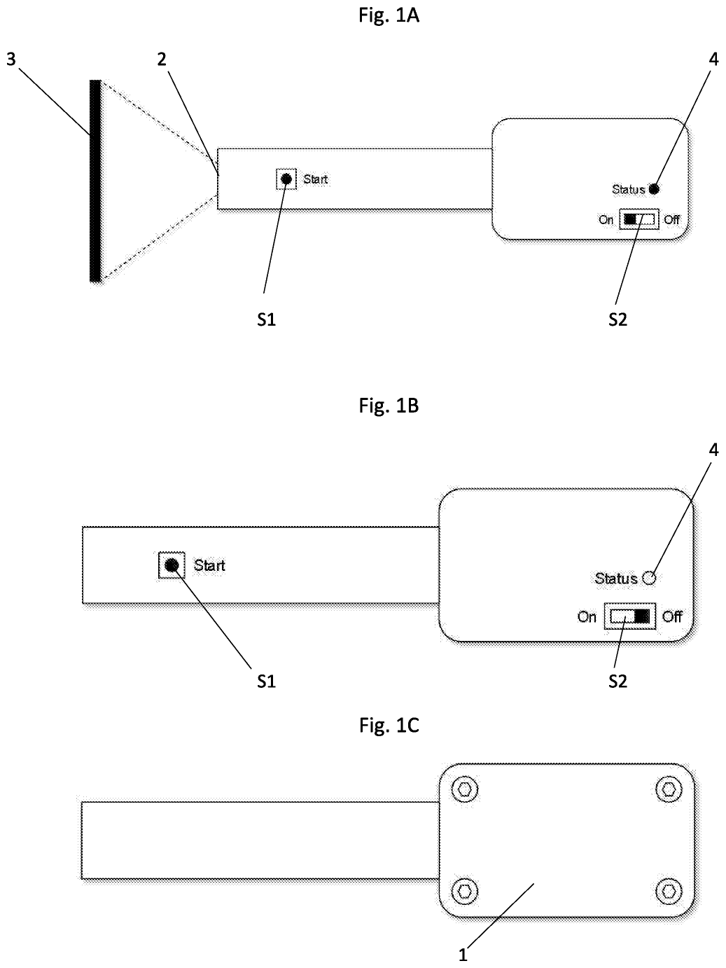

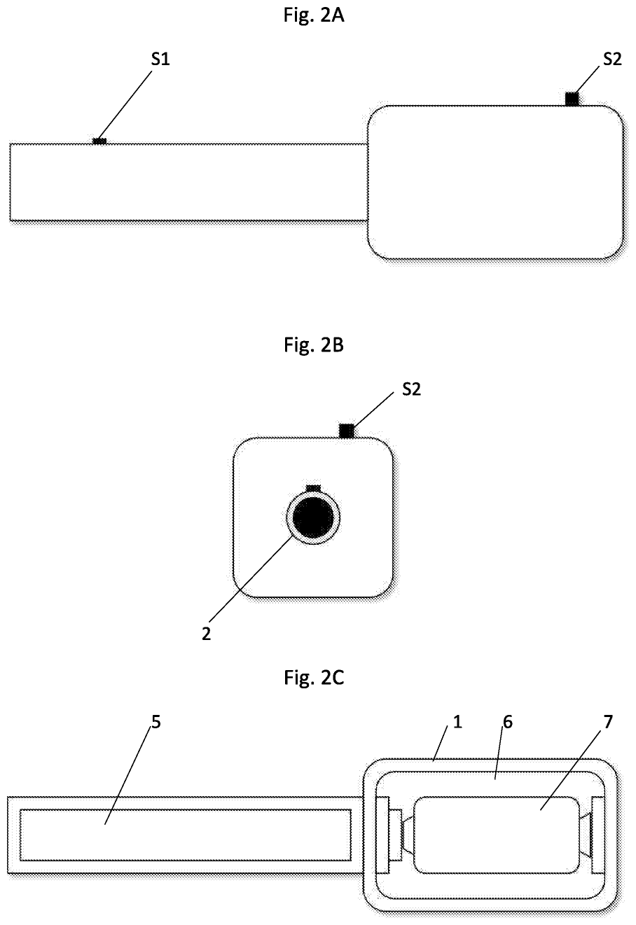

Referring to FIGS. 1A-1C, 2A and 2B, in a preferred embodiment of the present invention, the laser slit lamp comprises a housing 1 provided with a laser external window 2 (FIG. 2B) opened on the front end of the housing 1, through which the laser light is provided. The device can emit light to the ocular tissue of the patient through the laser external window to illuminate the patient's ocular tissue, and form a light straight line that creates a straight light line 3 at the ocular tissue of the patient. The device comprises an ON/OFF sliding switch S2 for energizing the circuit and, since the timing in the process is so critical, a START button S1 is placed on the front portion of the device so that the physician can activate it at the exact moment it is correctly aimed to the eye. A LED 4 placed next to the ON/OFF switch informs the user the status of the device, when LED is on indicates that laser is ready to use.

FIG. 2C shows that the device includes a laser light diode module 5 placed behind the laser external window 2, a timer circuit board 6 and a battery 6A. When the eye examination is performed, the switch S2 is turned on, the laser light diode module 5 generates the laser light that exits the device through the laser exit window 2 and the light beam is irradiated onto the patient's eye tissue to form a straight light line 3 in the eye. The effect that allows the laser diode to form a light straight line is generated inside the laser light module 5.

In this embodiment, the power source is preferably a 3.7 VDC (1000 mAH) rechargeable battery 6A, which is convenient for replacement.

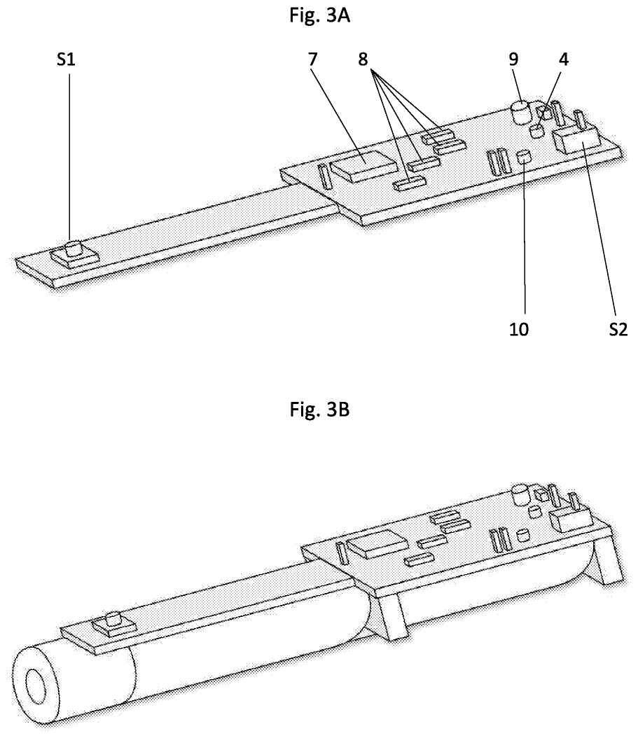

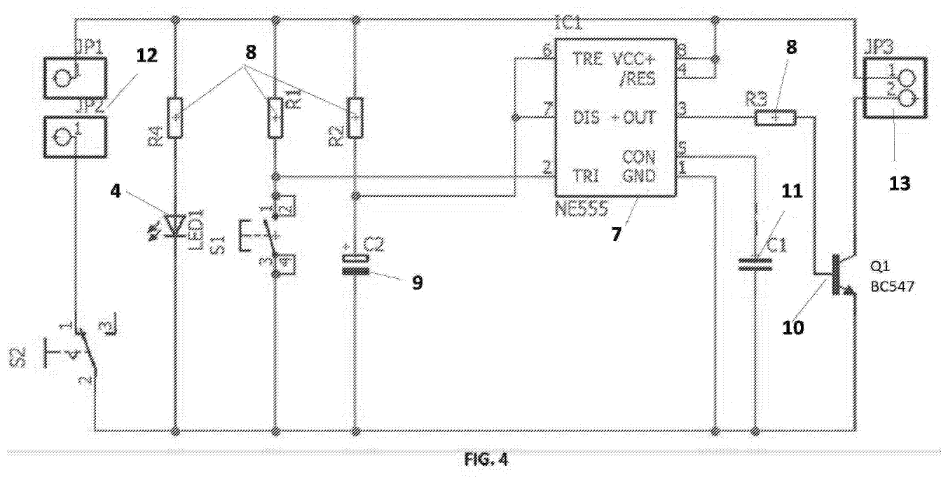

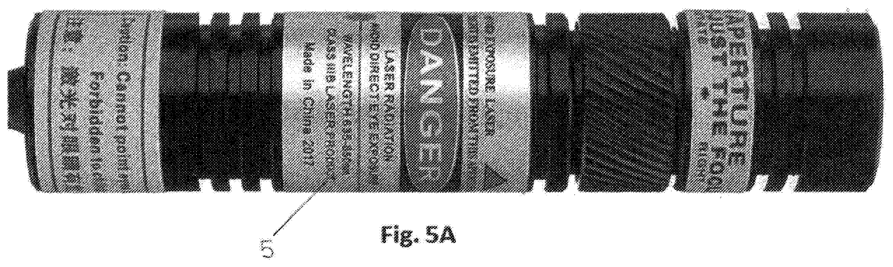

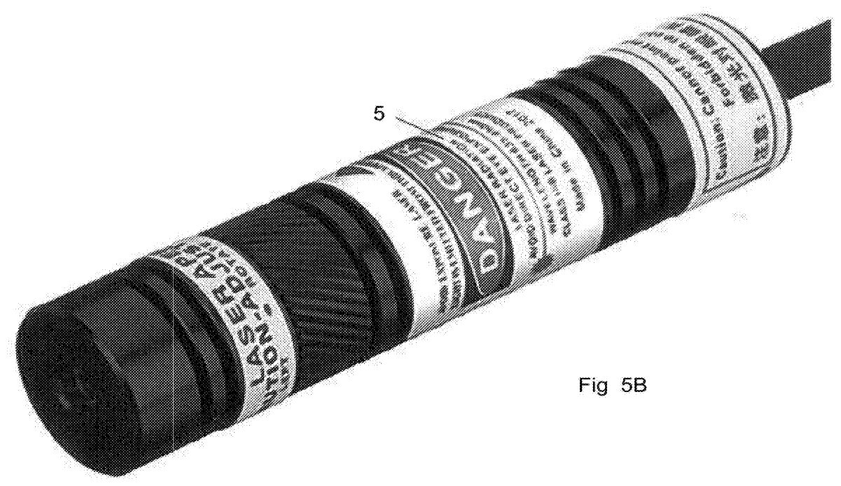

FIGS. 3A, 3B and FIG. 4 show that the timer circuit is formed by a printed circuit board 6 and the following electronic components soldered on it: (i) a timer IC 7 preferably model NE555 or similar; (ii) ¼ W carbon or metal film resistors (R1-R4) 8; (iii) an electrolytic capacitor 9; (iv) the status LED 4; (v) a NPN transistor 10 preferably model BC 547; (vi) a foil condenser 11; (vii) START push button S1; (viii) ON/OFF slide switch S2; jumpers JP1/JP2 12 and jumper JP3 13.

The operation of the timer and control board 6 is based on integrated circuit 555. It was configured as a monostable multivibrator in order to obtain a programmable delay to control the emission of laser light. The IC 555 timer is an integrated circuit that is used in the generation of timers, pulses and oscillations. The IC 555 can be used to provide time delays, as an oscillator, and as a flip flop chip. Its derivatives provide up to four synchronization circuits in a single package. When used as a monostable multivibrator the circuit delivers a single pulse of a width set by the designer, thus supplying the possibility of setting a programmable delay.

The power supply of device based on rechargeable battery 6A is controlled by S2 slide switch. When S2 is slid to ON position the status red LED turns on indicating that the system is energized and ready to use. Push button S1 is in charge of sending laser activation signal to the timer and control board.

The trigger signal of the 555 integrated circuit 7 is set to high level through pull up resistor R18; once S1 push button is pressed the signal changes to a low level state. The logic of the integrated circuit 7 reacts to this change of state sending to the output an activation pulse of predetermined duration.

The duration of the activation pulse is determined by discharge time of capacitive-resistive circuit and is given by the following equation T=ln (3)*R2*C2. In this case, values of electronic components R2 8 and C2 9 were selected in order to obtain a time delay of 60 seconds Finally, laser activation signal is sent to Q1 transistor 10 in order to adapt power level to manage the load that is connected to jumper JP3 13, namely the electric consumption of the laser diode module 5.

The illumination laser source is a laser diode module 5 supplied by third parties with the following specifications:

| Laser | Class IIIB semiconductor diode | |
|---|---|---|
| Max. Power | 100 | Mw |
| Wavelength | 650 nm/532 nm/405 nm | |
| Type of emission | Visible | |
| Emission shape | Line | |
| Pulse duration | 60 | s |
| Focal distance | 30 | mm |
| Dimensions | 1.1 cm × 3 mm × 3 mm | |
| Weight | 100 | g |

| | |
|---|---|
| Supply voltaje | 3.7 V (DC) |
| Temperature of op. | (−10 a 40)° C. |
| Expected lifetime | 5000 hours |

Laser module 5 comprises a heat dissipation housing 15 that contains the laser diode control board 16, a semiconductor laser diode 17, a fixed focusing lens 19 and a line generator lens 20.

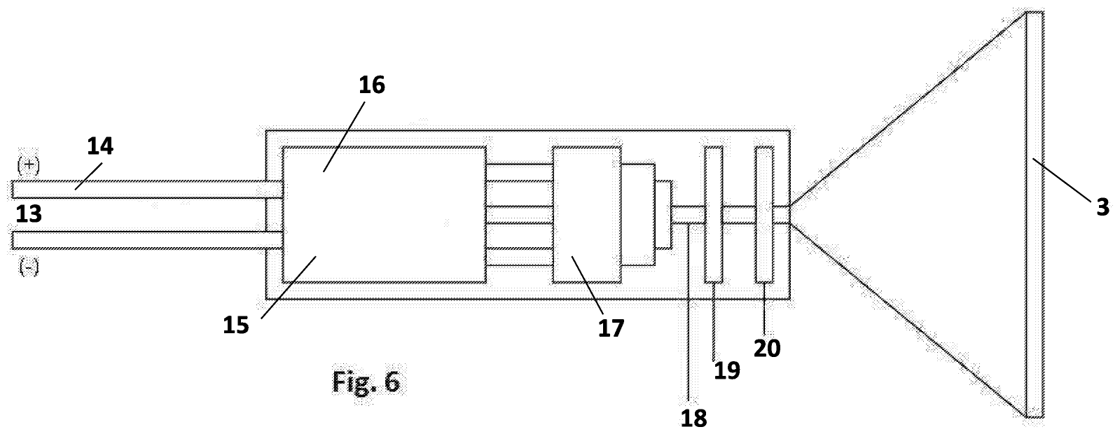

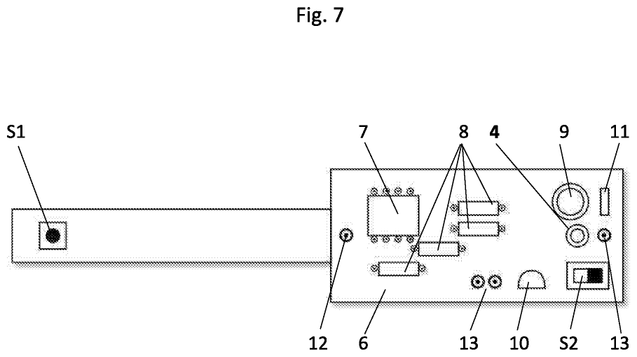

FIG. 6 shows that, when electrical power is outputted via jumper JP3 13, it is transmitted via power interface 14 to the laser diode control board 16. The board 16 energizes the laser diode 17 which emits a highly concentrated and bright laser beam 18, that is sent through collimator focusing lens 19 that adjusts the focal length to a fixed predetermined value, and the beam then passes through the line generator lens 20 that changes the shape of the laser light into a laser light straight line 3. The fixed focus is an important feature of the present invention since it allows that the surgeon varies the distance between the device and the inspected tissue to optimize the laser line projection. In a preferred embodiment, the fixed predetermined focal length is set at 30 mm.

Once the device is activated, the physician must carefully aim the laser straight line forming an angle of 30° to 50°, but not greater to avoid interfering the light with his hand. Also, keeping the angle between 30° and 50°, the laser light will not touch the macula, which is the most sensitive area of the retina. The timer comprised in the device will keep the eye exposure to laser impingement well below safety periods. A period of 60 seconds should be considered a maximum time limit for this exposure. The color of the laser beam can be violet, green or red, but the latter is the one best suited for visualizing abnormalities and it has proven to be less damaging for the eye.

Using a laser straight line for inspecting the eye instead of using a small circular LED or halogen spot is the heart of the present invention and has shown to have great advantages such as:
the laser line of the present invention is narrow, has always the same size and the surgeon only needs to find the exact focus for the perfect optical cut;
the device of the present invention shows a high brightness laser light emission;
the device of the present invention has a wider illumination span and high sharpness beam;
the device of the present invention allows to show high quality and definition cornea images;
the device of the present invention allows fastest examinations; and
the device of the present invention is easy to focus on ocular tissue.

In another aspect the present invention comprises a method of eye inspection which is based on the usage of the device of the present invention. In a preferred embodiment the method may comprise a method for detecting surgical ophthalmic abnormalities.

In a first preferred embodiment, the method of the present invention for detecting surgical ophthalmic abnormalities comprises scroll positioning verification (correct placement of the donor graft) during DMEK (Descemet membrane endotheilial kerastoplasty) surgery using the device of the present invention after using an eye inspection microscope.

The method comprises the following steps after turning off the microscope and ambient lights:
a—Turning on the portable device and activating the safety timer;
b—aiming the laser line of the portable device to the donor eye tissue to be inspected;
c—focusing on donor eye tissue and finding the correct angle of incidence of the laser beam in order to obtain a clear image;
d—verifying the correct position of the graft scrolling (orientation of the donor graft); and
e—interpreting the projected image seen inside the eye.

Steps a and b are quite straightforward. However, when the device is turned on in step a, the safety timer is automatically activated and this is one of the novel features of the present invention since it guarantees that the patient's eye is protected against irradiation overload.

Step c comprises forming an angle of 30° to 50° with the laser beam respect of a line which is perpendicular to the plane of the eye, but not greater, to avoid interfering the light with the surgeon's hand.

Step d is essential when projecting a laser light line inside the eye. DMEK surgery is a laminar corneal graft operation in which only Descemet's membrane and endothelium are replaced. During this procedure, at the moment of implantation and injection of the donor graft, the graft takes a double scroll shape. The graft scrolling in DMEK surgery refers to the tendency that donor eye tissues have, for being usually very thin, to naturally roll-up over themselves, with the endothelial at the outer side. Therefore, it is very important to check the orientation of donor tissue in order to keep the cornea structure unchanged and avoid reversing the order of cornea layers. If this issue is detected, the surgeon must change the orientation of the donor tissue, otherwise the scroll being oriented upside down will result in a transplant failure.

The endothelium must be in contact with aqueous humor and, therefore, if the scroll is attached upside down (e.g. the endothelium in touch with the stroma instead of the aqueous humor) it will detach in 3 days and the transplant will surely fail.

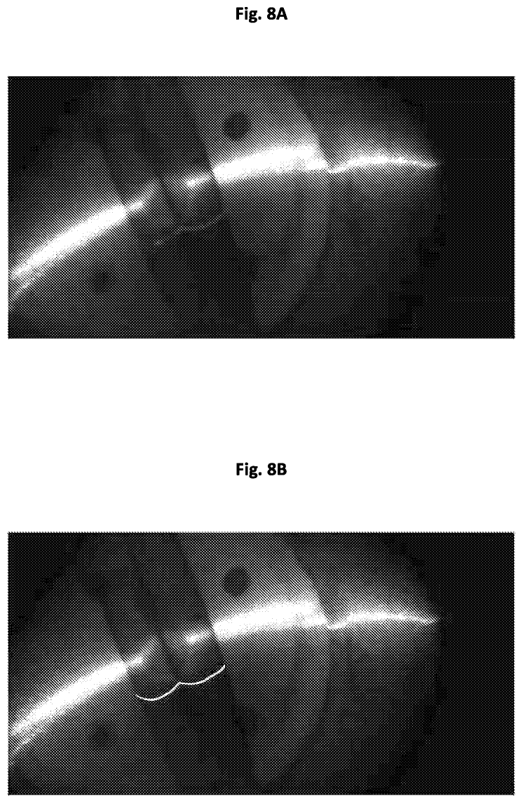

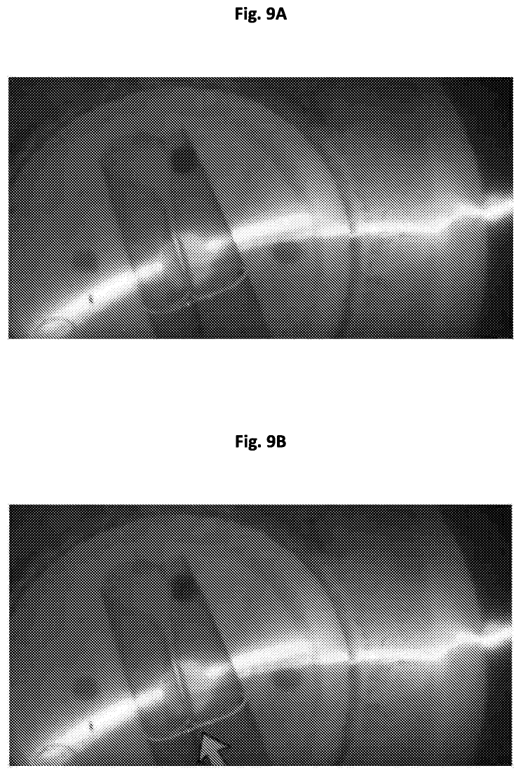

FIGS. 8A-8B show a preferred embodiment of the method when applied on a cornea. During examination, if it is possible to clearly see a number “3” formed by the laser line on the cornea (FIG. 8B), the surgeon can be sure of the correct positioning of the graft scroll. However, as may be seen in FIGS. 9A-9B, if the surgeon sees a letter “C” formed by the laser light (FIG. 9B), this indicates that the graft scroll is reversed.

Other real life cases testing the device in action and in relation to the correct scrolling may be seen in the following figures:

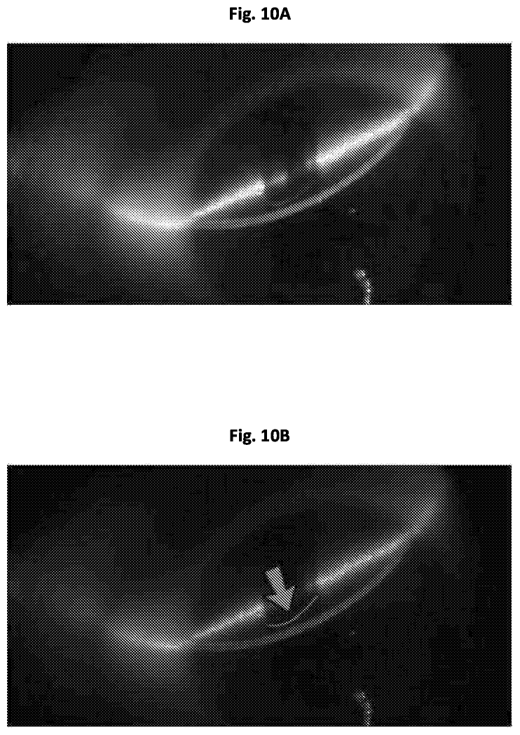

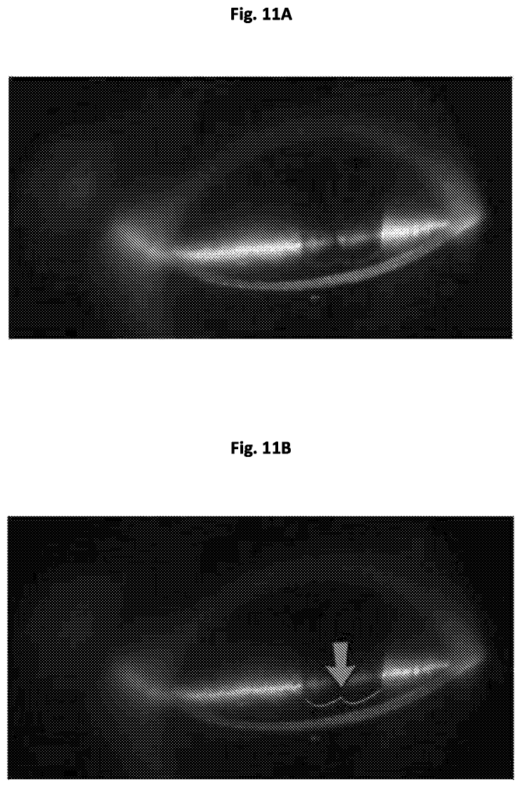

FIGS. 10A-10B and 11A-11B. FIG. 10B shows a situation in which a “C” may be seen in which the scrolling is upside down, while FIG. 11B shows a case (shown by the number “3”) in which the scrolling is correct.

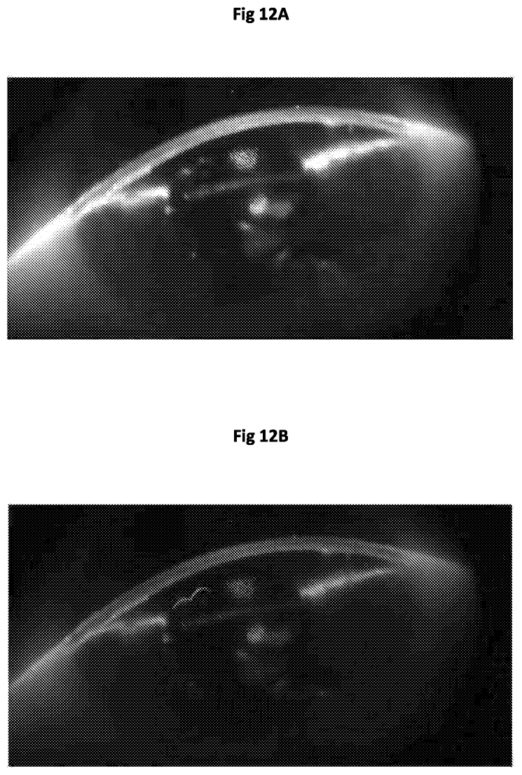

FIGS. 12A-12B show an edematous cornea in which the scrolling is correct, thus showing the number “3” in FIG. 12B.

Once the scroll positioning is verified the surgeon can further inspect the status of the patient's eye.

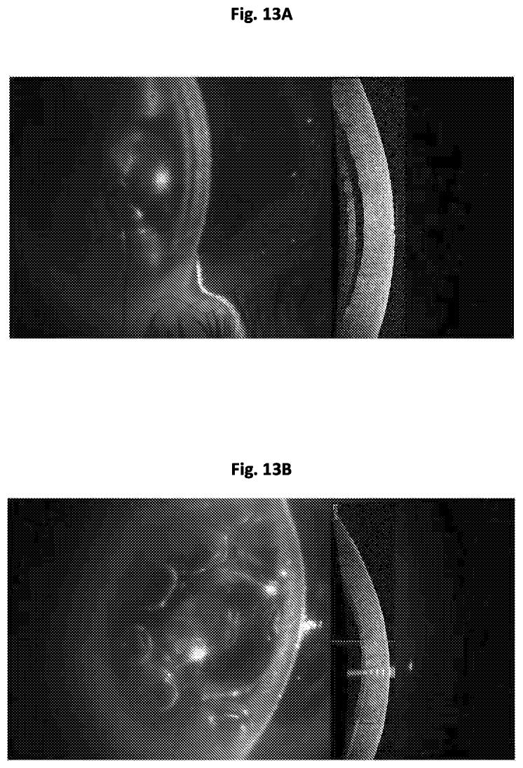

Step f is the beneficial result of using a laser light line instead of a laser light dot. This difference is the main core of the present invention and can be clearly understood when looking the photos of FIGS. 13A to 16C. In these, it may be clearly seen that the information obtained by the method is repetitive and does not require further knowledge of the detected eye abnormality.

FIGS. 13A-13B show a graft attachment verification after DSAEK (Descemet's Stripping Automated Endothelial Keratoplasty) surgery. In these procedures the donor tissue is thicker and it is prepared by cutting it with an apparatus and, therefore, it does not tend to roll-up. Hence, no graft scrolling is expected. However, when the eye gets softer after the intervention, the issue that may happen is a graft detachment and this may be seen in a gap between the cornea back face and the donor graft. When using the laser device, it may be seen in FIG. 13A that the graft is not completely attached when seeing the gap formed therein. On the left side of the figure an OCT (Optical coherence tomography) image confirms the graft detachment. In FIG. 13B it may be seen the result of laser examination after an air injection into the eyeball anterior chamber. If this issue is detected, the surgeon must inflate the anterior segment of the eye with air or a special gas, to keep the donor tissue attached.

The laser device of the present invention provides a high quality and detail level image of the cornea that allows the surgeon to differentiate the layers of ocular tissue in order to confirm the attachment or detachment of the donor graft. The donor graft is completely attached when no gap is seen between the posterior stroma and the donor tissue. If a gap is present the surgeon can confirm the detachment and this can only be clearly seen if the area is illuminated with a laser light line but not with a light or laser dot.

The present invention can also be applied to other surgical ophthalmic checks which do not imply donor tissue grafting. In these applications the method consists of the following steps after turning off the microscope and ambient lights:
a. —turning on the portable device and automatically activating the safety timer;
b. —aiming the laser line of the portable device to the eye tissue to be inspected;
c. —focusing on eye tissue and finding the correct angle of incidence of the laser beam in order to obtain a clear image; and
d1. —interpreting the projected image seen inside the eye.
   Some examples of these checks are the following:

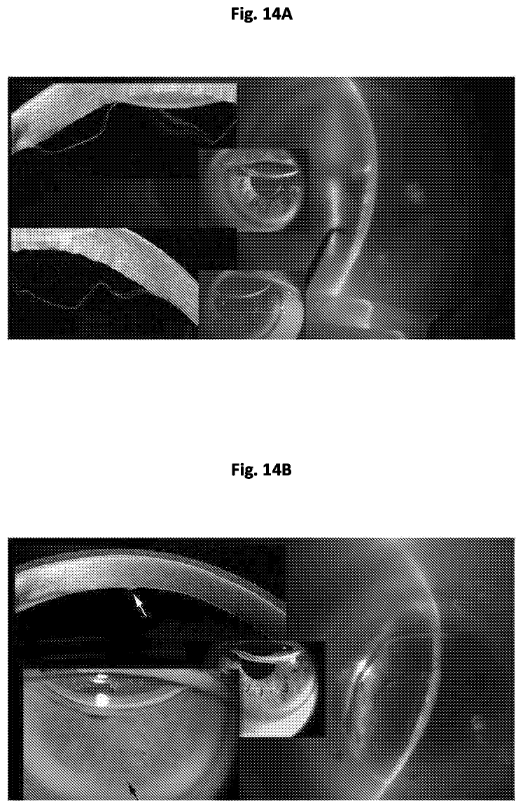

FIGS. 14A-14B show a case of Descemet membrane detachment detection during a chronic detachment surgery. Using the laser device of the present invention the surgeon clearly sees the cross section of the cornea where it is possible to check if there is a gap that shows the attachment or detachment of the Descemet membrane. When the surgeon needs to see a profile it is necessary to project a light or laser line that is reflected on the eye surface. It is impossible to see a profile with only one light or laser dot.

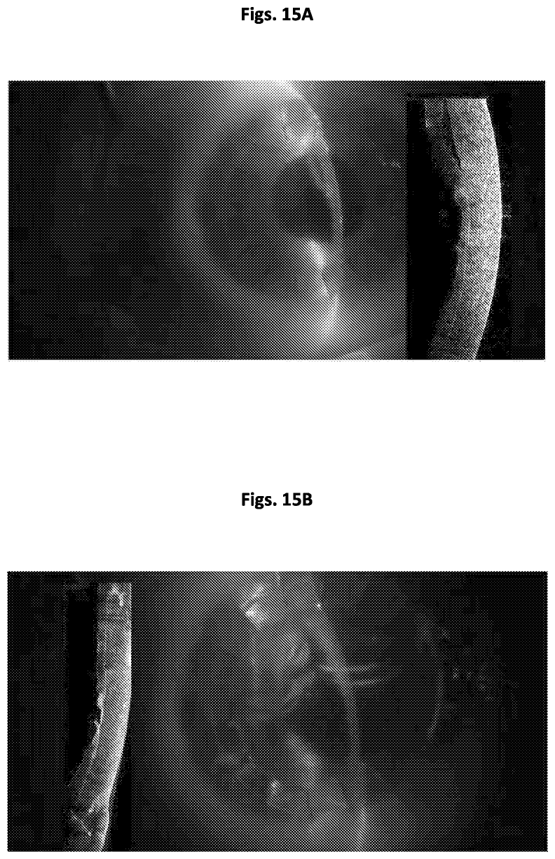

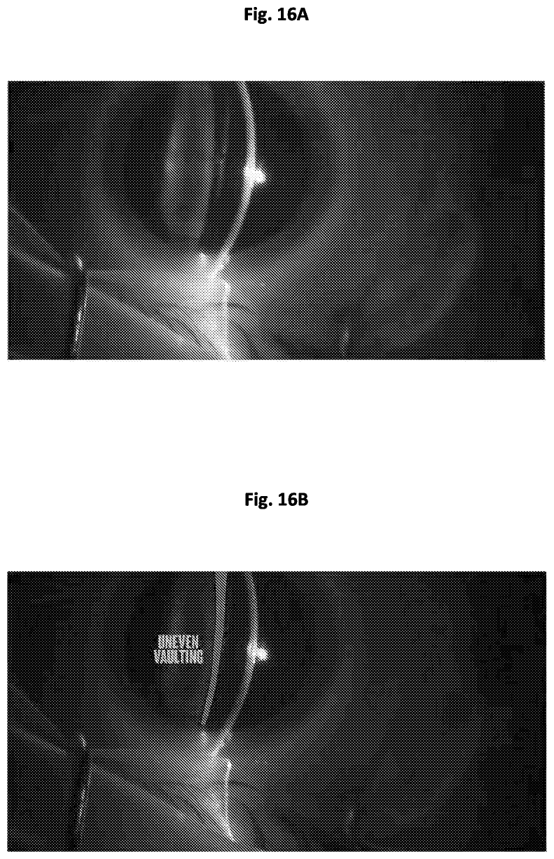

FIGS. 15A-15B show a case of Descemet membrane detachment detection after a DALK (Deep Anterior Lamellar Keratoplasty) surgery. When using the laser device of the present invention, here again the surgeon clearly sees the cross section of the cornea where it is possible to check the attachment or detachment of the Descemet membrane. The line laser creates a profile of the detached membrane and the other overlying surfaces of the cornea, making possible to see the gap between both surfaces. This would be impossible with a laser that emits only a spot because a spot does not create a profile.

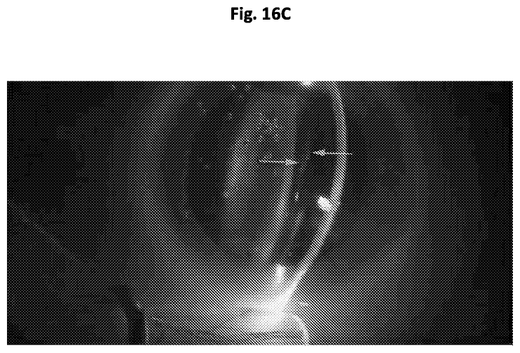

FIGS. 16A-16C illustrate an ICL (Implantable contact lens) vaulting verification in which an uneven vaulting detection may be obtained (FIG. 16B). FIG. 16C shows the ICL vaulting check after the correction when rotating 90 degrees the lens. An implantable contact lens is an artificial lens which is implanted in front of the eye's natural lens and behind the iris. During this procedure the laser device of the present invention allows the surgeon to take a qualitative measure of the distance between natural lens and iris in order to check lens positioning or vaulting. The fact that a light or laser line allows the surgeon to see a light profile makes it possible to compare the relative distance of the posterior surface of the Implantable Contact Lens in relation with the anterior surface of the human lens. The emission of a light or laser spot does not allow creating this profile.

### ANNEX

### Reference Element

1 Housing
2 Laser external window
3 Laser straight line
4 Status LED
5 Laser module
6 Timer circuit board
6A Rechargeable battery
7 Timer IC 555
8 ¼ W Resistor
9 Electrolytic capacitor
10 NPN transistor
11 Foil condenser
12 Jumper JP1/JP2
13 Jumper JP3
14 Power interface
15 Heat dissipation housing
16 Laser diode control board
17 Laser diode
18 Laser beam
19 Focusing lens
20 Line generator lens
S1 Start/stop button
S2 ON/OFF sliding switch

1. An portable laser device for ophthalmic examination, comprising a portable housing (1), a window (2) formed at the front end of said housing, an electric power supply (6A), a laser diode module (5) for laser emission, a status LED (4), a start push button (S1) and an ON/OFF sliding switch (S2);
wherein said housing (1) further contains a timer control board (FIGS. 3A, 3B and 4) for limiting the time period of laser emission to a predefined safety time value; and
wherein said laser emission of said laser diode module (5) is in form of a laser straight line.

2. The portable laser device according to claim 1, wherein said electric power supply comprises a 3.7 VDC (1000 mAH) rechargeable battery (6A).

3. The portable laser device according to claim 1, wherein said laser diode module (5) comprises a heat dissipation housing (15) that contains a laser diode control board (16), a semiconductor laser diode (17), a fixed focusing lens (19) and a line generator lens (20).

4. The portable laser device according to claim 3, wherein the fixed focusing lens fixes the focal length at 30 mm.

5. The portable laser device according to claim 1, wherein said timer control board comprises a printed circuit board (6); a timer IC (7); 4 resistors (8); an electrolytic capacitor (9); said status LED (4); a transistor (10); a foil condenser (11); said START push button (S1); said ON/OFF slide switch (S2); and connection jumpers (12; 13).

6. The portable laser device according to claim 5, wherein said IC timer (7) is configured as a monostable multivibrator,

7. The portable laser device according to claim 5, wherein said resistors (8) are ¼ W carbon or metal film resistors.

8. The portable laser device according to claim 5, wherein said transistor (10) is NPN type.

9. The portable laser device according to claim 5, wherein the value of said safety time period of laser emission is determined by discharge time of a capacitive-resistive circuit given by the following equation T=ln (3)*R2*C2.

10. The portable laser device according to claim 9, wherein the values of resistor R2 (8) and capacitor C2 (9) are selected to obtain a safety time period of 60 seconds.

11. A method of eye inspection carried out by a user by means of a portable laser device, the device comprising: a portable housing (1), a window (2) formed at the front end of said housing, an electric power supply (6A), a laser diode module (5) for laser emission, a status LED (4), a start push button (S1) and an ON/OFF sliding switch (S2); wherein said housing (1) further contains a timer control board (FIGS. 3A, 3B and 4) for limiting the time period of laser emission to a predefined safety time value; and wherein said laser emission of said laser diode module (5) is in form of a straight laser line beam;
wherein the method is for detecting surgical ophthalmic abnormalities.

12. The method according to claim 11, wherein said method is for detecting surgical ophthalmic abnormalities of donor eye tissue graft and comprising the steps of:
a. —turning on the portable device and automatically activating the safety timer;
b. —aiming the laser line of the portable device to a donor eye tissue to be inspected;
c. —focusing on the donor eye tissue and finding the correct angle of incidence of the laser line beam in order to obtain a clear image;
d. —verifying the correct position of the graft scrolling (orientation of the donor graft); and
e. —interpreting the projected image seen inside the eye.

13. The method according to claim 12, wherein said correct angle of incidence of the laser line beam is within a range of 30° to 50° respect of a line perpendicular to the eye.

14. The method according to claim 12, in which, after carrying out steps b and c, if said position of the graft scrolling is correct, in step d the laser line beam forms on the inspected tissue a number “3” or if said position of the graft scrolling is reversed the laser line beam forms on the inspected tissue a letter “C”.

15. The method according to claim 12, wherein the method comprises a graft attachment verification after DSAEK (Descemet's Stripping Automated Endothelial Keratoplasty) surgery involving eye posterior stroma and wherein step e comprises seeing that no gap exists between the posterior stroma and the donor tissue when the donor graft is completely attached.

16. The method according to claim 11, wherein the method comprises Descemet membrane detachment detection during a chronic detachment surgery and comprising the steps of:
a. —turning on the portable device and automatically activating the safety timer;
b. —aiming the laser line beam of the portable device to the eye tissue to be inspected;
c. —focusing on eye tissue and finding the correct angle of incidence of the laser line beam in order to obtain a clear image; and
d1. —interpreting the projected image seen inside the eye;
wherein in step c the laser line forms a profile of the eye surface, by which in step d1 the user may see the cross section of the cornea where it is possible to check the existence of a gap which shows a detachment of the Descemet membrane.

17. The method according to claim 11, wherein the method comprises Descemet membrane detachment detection in a DALK (Deep Anterior Lamellar Keratoplasty) surgery and comprising the steps of:
a. —turning on the portable device and automatically activating the safety timer;
b. —aiming the laser line beam of the portable device to the eye tissue to be inspected;
c. —focusing on eye tissue and finding the correct angle of incidence of the laser beam in order to obtain a clear image; and
d1. —interpreting the projected image seen inside the eye;
wherein the line laser beam creates in step c a profile of the Descemet membrane and of the other overlying surfaces of a cornea, by which the user sees in step d1 the cross section of the cornea thus allowing him to see a gap between both surfaces if the Descemet membrane is detached.

18. The method according to claim 11, wherein the method comprises Implantable contact lens (ICL) positioning or vaulting verification, further implying a human lens and a human iris, the method comprising the steps of:
a. —turning on the portable device and automatically activating the safety timer;
b. —aiming the laser line beam of the portable device to the eye tissue to be inspected;
c. —focusing on eye tissue and finding the correct angle of incidence of the laser line beam in order to obtain a clear image; and
d1. —interpreting the projected image seen inside the eye;
wherein the laser line beam forms a profile of the Implantable Contact Lens by which the user takes in step c a qualitative measure of the distance between the human lens and the iris in order to check in step d1 the lens positioning or vaulting, comparing the relative distance of the posterior surface of the Implantable Contact Lens in relation with the anterior surface of the human lens.
